(19) United States
(12) Patent Application Publication
(10) Pub. No.: US 2016/0021839 A1
(43) Pub. Date: Jan. 28, 2016

(54) APPARATUS AND METHOD FOR MANUAL DISPENSING OF GRAINS OF POLLEN

(71) Applicant: PIONEER HI-BRED INTERNATIONAL, INC., Johnston, IA (US)

(72) Inventors: AMY DIANE BLAHNIK, Johnston, IA (US); GARY LANE JAEHNEL, Des Moines, IA (US)

(73) Assignee: PIONEER HI-BRED INTERNATIONAL, INC., JOHNSTON, IA (US)

(21) Appl. No.: 14/773,986

(22) PCT Filed: Mar. 11, 2014
(86) PCT No.: PCT/US2014/023627

§ 371 Date: Sep. 9, 2015

Related U.S. Application Data
(60) Provisional application No. 61/792,105, filed on Mar. 15, 2013.

(51) Int. Cl. A01H 1/02; B05B 7/14

(52) U.S. Cl. CPC A01H 1/025; B05B 7/1486

(57) ABSTRACT

An apparatus for controlled pollination of a maize plant is provided. The apparatus may include a reusable air discharger having an internal cavity and an outlet, and configured, when actuated, to expel air from the internal cavity through the outlet and into a single use, biodegradable applicator having a first open end and a second open end. The applicator may further be configured to receive at least one maize tassel containing maize pollen thereon, and to direct the maize pollen through the second end of the applicator upon actuation of the air discharger. A method for controlled pollination of two or more maize plants using a common air discharger is also provided.

### FIELD OF THE INVENTION

Various embodiments of the present invention relate generally to methods and apparatuses for manual dispensing of pollen. More specifically, embodiments of the present invention provide a method and apparatus configured to facilitate manual dispensing of grains of pollen from one or more tassels.

### BACKGROUND OF THE INVENTION

For a variety of reasons, plant species may be intentionally bred. For example, in some applications plant species are intentionally bred to form hybrid plant species. In some applications, hybrid plants are bred to exhibit various desirable traits. Such traits may include, for example, resistance to heat and drought, resistance to disease and insect damage, improved yield characteristics, and improved agronomic quality. In general, plants may be capable of self-pollination, cross-pollination, or both. Self-pollination describes pollination using pollen from one flower that is transferred to the same or another flower of the same plant. Cross-pollination describes pollination using pollen delivered from a flower of a different plant from a different family or line.

Plants that have been self-pollinated and selected for many generations become homozygous at almost all gene loci and produce a uniform population of true breeding progeny. A cross between two different homozygous lines produces a uniform population of hybrid plants that may be heterozygous for many gene loci. A cross of two plants each heterozygous at a number of gene loci will produce a population of heterogeneous plants that differ genetically and will not be uniform.

Maize (Zea mays L.), often referred to as corn in the United States, can be bred by both self-pollination and cross-pollination techniques. Maize has separate male and female flowers on the same plant. The male flowers are located on the tassel and the female flowers are located on the ear. Natural pollination occurs in maize when wind blows grains of pollen from the tassels to the silks that protrude from the tops of the ears.

The development of a hybrid maize variety in a maize seed production program may involve three steps: (1) the selection of plants from various germplasm pools for initial breeding crosses; (2) self-pollination of the selected plants from the breeding crosses for several generations to produce a series of inbred lines, which, individually breed true and are highly uniform; and (3) crossing a selected inbred line with an unrelated inbred line to produce the hybrid progeny. After a sufficient amount of inbreeding successive filial generations will merely serve to increase seed of the developed inbred. Preferably, an inbred line should comprise homozygous alleles at about 95% or more of its loci.

During the maize inbreeding process, vigor of the line may decrease. Vigor may be restored when two different inbred lines are crossed to produce the hybrid progeny. An important consequence of the homozygosity and homogeneity of the inbred lines is that the hybrid between a defined pair of inbreds may be reproduced indefinitely as long as the homogeneity of the inbred parents is maintained. Once the inbreds that create a superior hybrid have been identified, a continual supply of the hybrid seed can be produced using these inbred parents and the hybrid corn plants can then be generated from this hybrid seed supply.

Accordingly, development and production of maize seed may require controlled pollination at one or more steps, as described above.

### BRIEF SUMMARY

In one embodiment an apparatus for manual dispensing of maize pollen is provided. The apparatus includes a reusable air discharger having an internal cavity and an outlet, the air discharger being configured, when actuated, to expel air from the internal cavity through the outlet. The apparatus also includes a single use, biodegradable applicator having a first open end and a second open end, wherein the first open end is in communication with the outlet of the air discharger. The applicator is further configured to receive at least one maize tassel containing maize pollen thereon, and to direct the maize pollen through the second end of the applicator upon actuation of the air discharger.

In another embodiment, the applicator may comprise a cone made of a paper material, and the first open end of the applicator may comprises a larger open end of the cone and the second open end of the applicator may comprise a smaller open end of the cone. In another embodiment, the air discharger may comprise a hand-operated bulb pump that further comprises an inlet, and wherein, when actuated, the bulb pump may be further configured to introduce air into the internal cavity through the inlet. In one embodiment, the bulb pump further comprises two one-way valves, one of which is in communication with the inlet and the other of which is in communication with the outlet, such that, when actuated, air expelled from the internal cavity only through the outlet, and is introduced into the internal cavity only through the inlet, thus creating unidirectional air flow. In another embodiment, the air discharger may include indicia configured to indicate a proper orientation between the air discharger with the applicator.

### BRIEF DESCRIPTION OF THE SEVERAL VIEWS OF THE DRAWING(S)

Having thus described the invention in general terms, reference will now be made to the accompanying drawings, which are not necessarily drawn to scale, and wherein:

FIG. 1 illustrates an embodiment of an apparatus configured for manual dispensing grains of pollen from one or more tassels in accordance with an example embodiment of the present invention;

FIG. 2 illustrates a view of some of the components of the embodiment of FIG. 1; and

FIG. 3 illustrates a method for controlled pollination of two maize plants in accordance with an example embodiment of the present invention.

### DETAILED DESCRIPTION

Manually collecting and dispensing pollen using existing methods may be very labor intensive. For example, using existing methods a worker may manually collect pollen by knocking it off of the tassels of the ear corn into a tassel bag. Further, the worker may then manually sprinkle the pollen onto the anthers of female corn which have been covered with a shoot bag so as to prevent pollination with pollen other than the collected grains of pollen. Collection of pollen and controlled pollination using existing methods may involve many manual steps that may be conducted over the course of one or more days. Further, application of pollen using tassel bags may not efficiently make use of the collected pollen since the tassel bag may not allow for targeted application of the pollen in an easily controlled manner. Thus, workers may tend to apply more of the pollen than would otherwise be necessary. Accordingly, embodiments of the invention provide improved apparatuses and methods for manual dispensing pollen.

The present invention now will be described more fully hereinafter with reference to the accompanying drawings, in which some, but not all embodiments of the invention are shown. Indeed, the invention may be embodied in many different forms and should not be construed as limited to the embodiments set forth herein; rather, these embodiments are provided so that this disclosure will satisfy applicable legal requirements. Like numbers refer to like elements throughout.

According to the present invention, controlled pollination of plants, for example during cross-pollination, involves collecting and dispensing pollen using the apparatuses and methods described herein.

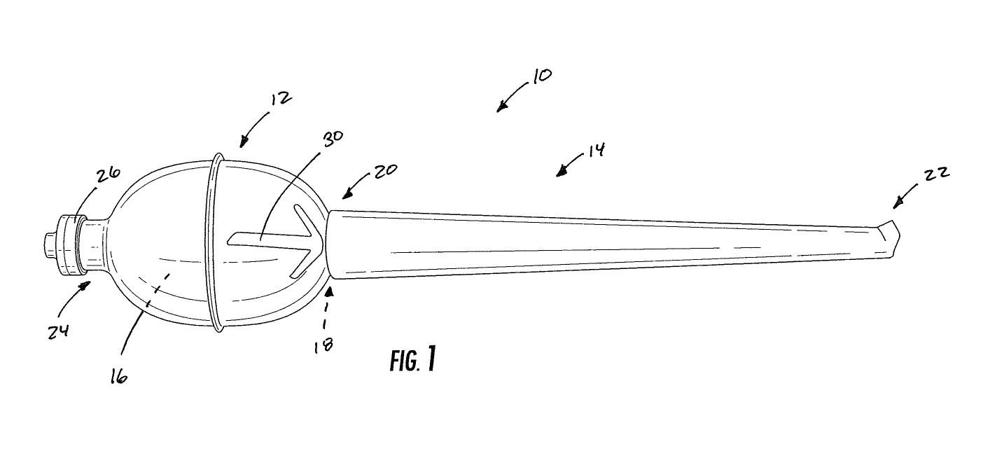

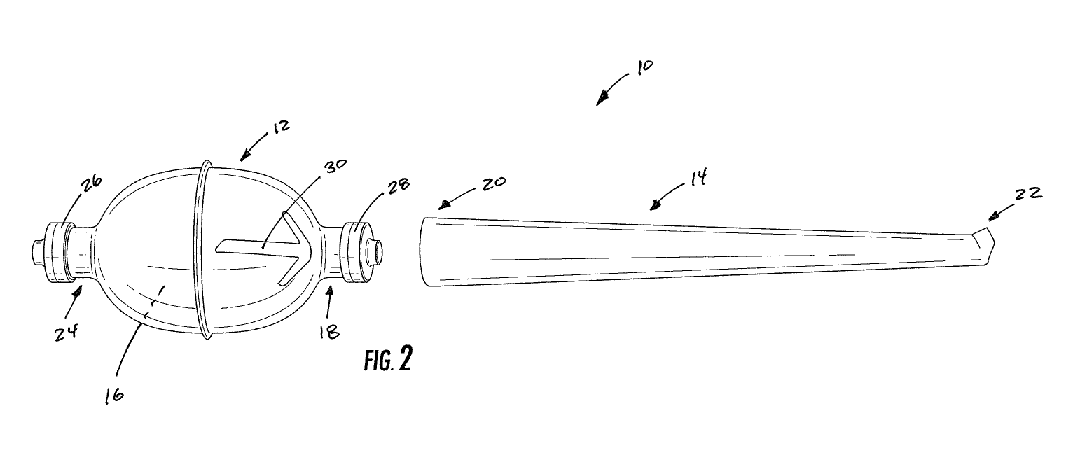

In this regard, FIGS. 1 and 2 illustrate embodiments of an apparatus 10 configured for manual dispensing grains of pollen. The apparatus 10 may comprise an air discharger 12, and an applicator 14. In the depicted embodiments, the air discharger 12 comprises an internal cavity 16, and an outlet 18, which is in communication with the internal cavity 16. Also in the depicted embodiments, the applicator 14 comprises a first open end 20, and a second open end 22, which is in communication with the first open end 20. The applicator 14 is further configured to receive at least one maize tassel containing maize pollen thereon.

When the apparatus 10 is in an assembled condition (FIG. 1), the outlet 18 is positioned proximate to the first open end 20 of the applicator 14 (which, if pollination is desired, contains at least one maize tassel containing maize pollen thereon) such that the outlet 18 of the air discharger 12 is configured to be in communication with the first open end 20 of the applicator 14. Upon actuation of the air discharger 12, air is propelled through the outlet 18 of the air discharger 12 and past the maize tassel. As the air passes the tassel, pollen is picked up by the air stream and is directed through the second open end 22 of the applicator. Because the applicator 14 has a funnel shape (in the depicted embodiment, the first open 20 end has a larger size (e.g., diameter) than that of the second open end 22), the generated pollen blast may be concentrated and thus may enable the user to accurately direct the pollen onto a target (such as, for example, the silks of a target maize plant).

Although in other embodiments the air discharger may be one of a variety of devices (for example, in some embodiments, the air discharger may be a single orifice device capable of discharging a gas, such as a CO₂ cartridge), in the depicted embodiment the air discharger 12 is a hand-operated bulb pump having, in addition to the outlet 18, an inlet 24. Upon being actuated, such as, for example, by squeezing the bulb, the hand-operated bulb pump is configured to expel air through the outlet 18. When the bulb is released, the hand-operated bulb pump is configured to draw air into its internal cavity 16 through the inlet 24. To aid in this operation, the hand-operated bulb pump of the depicted embodiment includes a first one-way valve 26, which is in communication with the inlet 24, and a second one-way valve 28, which is in communication with the outlet 18. In such a manner, when the hand-operated bulb pump is actuated, air is expelled from the internal cavity 16 only through the outlet 18, and is introduced into the internal cavity 16 only through the inlet 24, thus creating unidirectional air flow. It should be noted that in other embodiments, a semi-automated device could replace the bulb to generate the air stream. For example, a small battery powered air compressor could be activated, such as, for example, by pressing a button or pulling a trigger.

This unidirectional air flow is advantageous in controlling the source of pollen in a controlled pollination event when using the air discharger 12 to pollinate multiple target maize plants. To further aid in controlling the source of pollen in a controlled pollination event, indicia 30 may be added to indicate the proper mating orientation of the air discharger 12 with respect to the applicator 14. In the depicted embodiment, indicia 30 comprises an arrow that shows the direction of air flow through the air discharger 12. As such, in various embodiments multiple applicators 14 may be used, each applicator 14 configured to receive at least one maize tassel containing maize pollen thereon, together with a common air discharger 12. In various embodiments, the applicators 14 may be configured to be single use, biodegradable applicators. Although in various embodiments, a single use, biodegradable applicator may be constructed of one or more of a variety of materials, in one embodiment single use, the biodegradable applicators may be constructed of a paper material. In such a manner, after use the single use biodegradable applicator may be discarded, such as on the ground of the field containing the maize plants. Since air only flows in one direction through the applicator 14, and with proper orientation established between the air discharger 12 and applicator 14, the present invention may increase the certainty that a controlled pollination event using pollen from one source (such as, for example, a tassel from one maize corn plant) will not cross-contaminate a controlled pollination event using pollen from another source (such as, for example, a tassel from another maize corn plant).

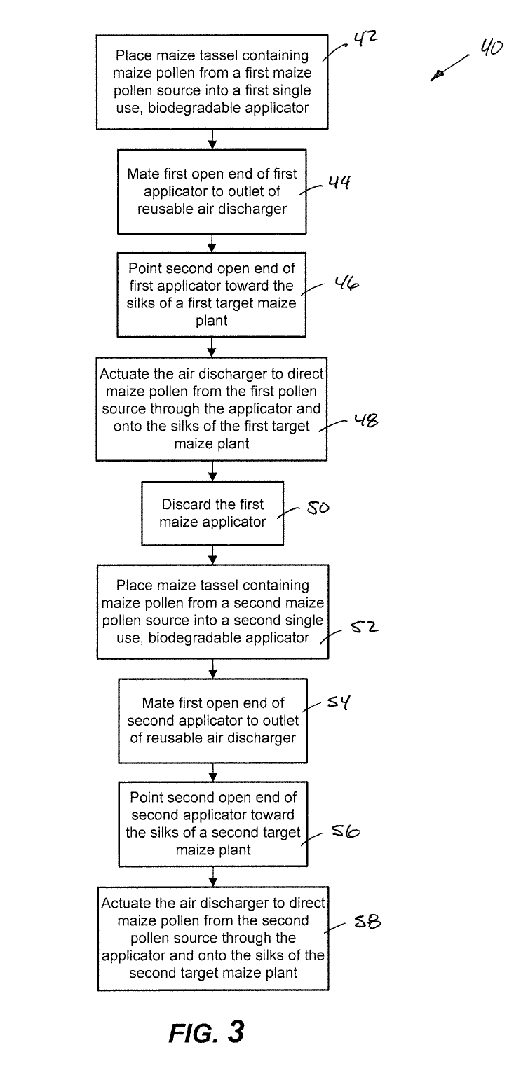

In such a manner, FIG. 3 illustrates a method for controlled pollination of two maize plants in accordance with an example embodiment of the present invention. Although FIG. 3 describes controlled pollination events occurring between two plants, the method of FIG. 3, or any portion or portions thereof, may be used for controlled pollination of a single maize plant or three or more maize plants. As shown in the figure, the method 40 may comprise placing at least one maize tassel containing maize pollen from a first maize pollen source thereon into a first single use, biodegradable applicator, in step 42; mating a first open end of the first applicator to an outlet of a reusable air discharger, in step 44; pointing a second open end of the first applicator toward the silks of a first target maize plant, in step 46; actuating the air discharger to direct the maize pollen of the first pollen source through the the first applicator and onto the silks of the first target maize plant, in step 48; discarding the first maize applicator, in step 50; placing at least one maize tassel containing maize pollen from a second maize pollen source thereon into a second single use, biodegradable applicator, in step 52; mating a first open end of the second applicator to an outlet of the reusable air discharger, in step 54; pointing a second open end of the second applicator toward the silks of a second target maize plant, in step 56; and actuating the air discharger to direct the maize pollen of the second pollen source through the second applicator and onto the silks of the second target maize plant, in step 58.

Although the above examples were made specific to maize plants, maize pollen, and maize tassels, the apparatuses, systems, and methods described herein may be applicable to any plant having similar tassel or pollen configurations.

Many modifications and other embodiments of the invention set forth herein will come to mind to one skilled in the art to which these invention pertains having the benefit of the teachings presented in the foregoing descriptions and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

That which is claimed:

1. An apparatus configured for manual dispensing of maize pollen, said apparatus comprising:
a reusable air discharger having an internal cavity and an outlet, and configured, when actuated, to expel air from the internal cavity through the outlet; and
a single use, biodegradable applicator having a first open end and a second open end, wherein the first open end is in communication with the outlet of the air discharger, and
wherein the applicator is further configured to receive at least one maize tassel containing maize pollen thereon, and to direct the maize pollen through the second end of the applicator upon actuation of the air discharger.

2. The apparatus of claim 1, wherein the applicator comprises a cone made of a paper material, and wherein the first open end of the applicator comprises a larger open end of the cone and the second open end of the applicator comprises a smaller open end of the cone.

3. The apparatus of claim 1, wherein the air discharger comprises a hand-operated bulb pump that further comprises an inlet, and wherein, when actuated, the bulb pump is further configured to introduce air into the internal cavity through the inlet.

4. The apparatus of claim 3, wherein the bulb pump further comprises two one-way valves, one of which is in communication with the inlet and the other of which is in communication with the outlet, such that, when actuated, air expelled from the internal cavity only through the outlet, and is introduced into the internal cavity only through the inlet, thus creating unidirectional air flow.

5. The apparatus of claim 4, wherein said one-way valves are configured to prevent contamination from entering the internal cavity.

6. The apparatus of claim 1, wherein the air discharger includes indicia configured to indicate a proper orientation between the air discharger with the applicator.

7. A method for manual dispensing of maize pollen, said method comprising:
placing at least one maize tassel containing maize pollen thereon into a single use, biodegradable applicator having a first open end and a second open end;
mating the first open end of the applicator to an outlet of a reusable air discharger having an internal cavity; and
actuating the air discharger such that air is expelled from the internal cavity past the maize tassel and through the second open end of the applicator, thus picking up and carrying at least some of the pollen of the maize tassel and directing the maize pollen through the second open end of the applicator.

8. The method of claim 7, wherein the applicator comprises a cone made of a paper material, and wherein the first open end of the applicator comprises a larger open end of the cone and the second open end of the applicator comprises a smaller open end of the cone.

9. The method of claim 7, wherein actuating the air discharger comprises actuating a hand-operated bulb pump that further comprises an inlet, and wherein, when actuated, the bulb pump is further configured to introduce air into the internal cavity through the inlet.

10. The method of claim 9, wherein the bulb pump further comprises two one-way valves, one of which is in communication with the inlet and the other of which is in communication with the outlet, such that, when actuated, air is introduced into the internal cavity only through the inlet and expelled from the internal cavity only through the outlet, thus creating unidirectional air flow.

11. The method of claim 10, wherein the one-way valves are configured to prevent contamination from entering the internal cavity.

12. The method of claim 7, wherein the air discharger includes indicia configured to indicate a proper orientation for mating the air discharger with the applicator.

13. A method for manual dispensing of two maize pollen sources onto the silks of two maize plants, said method comprising:
placing at least one maize tassel containing maize pollen from a first maize pollen source thereon into a first single use, biodegradable applicator having a first open end and a second open end;
mating the first open end of the first applicator to an outlet of a reusable air discharger having an internal cavity;
pointing the second open end of the first applicator toward the silks of a first target maize plant;
actuating the air discharger such that air is expelled from the internal cavity past the maize tassel of the first maize pollen source and through the second open end of the first applicator, thus picking up and carrying at least some of the pollen of the maize tassel of the first pollen source and directing the maize pollen of the first pollen source through the second open end of the first applicator and onto the silks of the first target maize plant;
discarding the first maize applicator;
placing at least one maize tassel containing maize pollen from a second maize pollen source thereon into a second single use, biodegradable applicator having a first open end and a second open end;
mating the first open end of the second applicator to the outlet of the reusable air discharger;
pointing the second open end of the second applicator toward the silks of a second target maize plant; and
actuating the air discharger such that air is expelled from the internal cavity past the maize tassel of the second maize pollen source and through the second open end of the second applicator, thus picking up and carrying at least some of the pollen of the maize tassel of the second pollen source and directing the maize pollen of the second pollen source through the second open end of the second applicator and onto the silks of the second target maize plant.

14. The method of claim 13, wherein each of the first and second applicators comprises a cone made of a paper material, and wherein the first open end of each respective applicator comprises a larger open end of the respective cone and the second open end of each respective applicator comprises a smaller open end of the respective cone.

15. The method of claim 13, wherein each instance of actuating the air discharger comprises actuating a common hand-operated bulb pump that further comprises an inlet, and wherein, when actuated, the bulb pump is further configured to introduce air into the internal cavity through the inlet.

16. The method of claim 15, wherein the bulb pump further comprises two one-way valves, one of which is in communication with the inlet and the other of which is in communication with the outlet, such that, when actuated, air is introduced into the internal cavity only through the inlet and expelled from the internal cavity only through the outlet, thus creating unidirectional air flow.

17. The method of claim 16, wherein the one-way valves are configured to prevent contamination from entering the internal cavity.

18. The method of claim 13, wherein the air discharger includes indicia configured to indicate a proper orientation for mating the air discharger with the applicator.
